(19) United States
(12) Patent Application Publication
(10) Pub. No.: US 2017/0143403 A1
(43) Pub. Date: May 25, 2017

(54) INSTRUMENTS AND METHODS FOR TREATING ULCERATIVE COLITIS AND OTHER INFLAMMATORY BOWEL DISEASES

(71) Applicant: COVIDIEN LP, Mansfield, MA (US)

(72) Inventors: WILLIAM H. NAU, JR., LONGMONT, CO (US); DUANE E. KERR, LOVELAND, CO (US); KATHY E. ROOKS, LONGMONT, CO (US); DARION R. PETERSON, LONGMONT, CO (US); ROBERT M. SHARP, BOULDER, CO (US); REBECCA J. COULSON, LYONS, CO (US)

(21) Appl. No.: 15/340,792

(22) Filed: Nov. 1, 2016

Related U.S. Application Data
(60) Provisional application No. 62/258,081, filed on Nov. 20, 2015.

(51) Int. Cl. A61B 18/08

(52) U.S. Cl. CPC A61B 18/082; A61B 2018/00047

(57) ABSTRACT

A surgical instrument includes a flexible tube defining a proximal end and a distal end. An end effector, disposed at the distal end of the flexible tube, is configured for insertion into and advancement through a body cavity to a treatment target. The end effector is adapted to connect to a source of energy. The end effector includes an expandable member which can expand from an un-expanded state to an expanded state in order to circumferentially contact a bodily treatment target. The end effector further includes at least one heating member that may hear the expandable member such that the expandable member may thermally treat tissue that the expandable member contacts.

### CROSS REFERENCE TO RELATED APPLICATION

The present application claims the benefit of and priority to U.S. Provisional Application Ser. No. 62/258,081, filed on Nov. 20, 2015, the entire contents of which are incorporated herein by reference.

### BACKGROUND

Technical Field

The present disclosure relates to treatment of Inflammatory Bowel Diseases (IBDs) and, more particularly, to surgical instruments and methods for treating ulcerative colitis and other IBDs, e.g., Crohn's Disease.

Background of Related Art

Ulcerative colitis, an IBD, is a disease of the colon in which inflammation and ulcers, or sores, form on the interior wall of the colon. Ulcerative colitis manifests itself, usually intermittently and at varying degrees of severity, in symptoms such as stomach pain, diarrhea, and/or bloody stool. Typically, anti-inflammatory medication(s) are prescribed for patients suffering from ulcerative colitis. In extreme cases, chronic cases, or cases in which medication(s) fails to adequately treat the patient's symptoms, surgery to remove all or part of the diseased portions of the rectum and/or colon may be performed.

Although medication(s) and surgical removal of diseased portions of the rectum and/or colon are effective in certain instances, there is a need for surgical instruments, systems, and methods to more effectively and/or efficiently treat ulcerative colitis and other IBDs, e.g., Crohn's Disease, while minimizing side effects and damage to un-diseased tissue.

### SUMMARY

As used herein, the term “distal” refers to the portion that is being described which is further from a user, while the term “proximal” refers to the portion that is being described which is closer to a user. Further, to the extent consistent, any of the aspects described herein may be used in conjunction with any or all of the other aspects described herein.

A surgical instrument provided in accordance with the present disclosure includes a flexible tube defining a proximal end and a distal end. An end effector, disposed at the distal end of the flexible tube, is configured for insertion into and advancement through a body cavity to a treatment target. The end effector is adapted to connect to a source of energy. The end effector includes an expandable member which can expand from an un-expanded state to an expanded state in order to circumferentially contact a bodily treatment target. The end effector further includes at least one heating member configured to heat the expandable member such that the expandable member is enabled to thermally treat tissue in contact therewith.

In an aspect of the present disclosure, the expandable member is an inflatable balloon. Further, the heating member may include a laser source disposed within the inflatable balloon. The laser source is configured to emit laser light onto the inflatable balloon and the inflatable balloon is configured to absorb the laser light, thereby heating the inflatable balloon.

In yet another aspect of the present disclosure, the expandable member is an inflatable balloon and the heating member is composed of one or more ultrasonic devices. The ultrasonic devices emit high frequency ultrasonic waves. The inflatable balloon is configured to absorb the ultrasonic waves, thereby heating the inflatable balloon.

In still another aspect of the present disclosure, the expandable member is an inflatable balloon and the heating member is a thermoelectric cooler. The thermoelectric cooler is disposed about the inflatable balloon.

In another aspect of the present disclosure, the expandable member is an inflatable balloon and the heating member is a conductive coating disposed on the expandable balloon. The conducting coating is configured to receive an electric current and generate heat through resistive heating.

In an additional aspect of the present disclosure, the expandable member is an expandable ring and the heating member includes a fluid chamber configured to heat fluid. The heated fluid is pumped into the expandable ring causing the expandable ring to expand and heat. As an expandable ring expands, it contacts a treatment site and transfers heat to the treatment site for thermal treatment thereof.

In yet another aspect of the present disclosure, the expandable member includes an expandable basket, and the heating member includes a plurality of wire components composing a portion of the expandable basket. The plurality of wire components is configured to receive electric current and produce heat through resistive heating. The expandable basket further includes a joint interconnecting at least two of the wire components. The joint is configured to flex and expand the expandable basket in response to energy conduction through the joint.

A method of treating tissue is also provided in accordance with the present disclosure. The method includes inserting a flexible tube having an end effector disposed at a distal end thereof into a body cavity, positioning the end effector adjacent a treatment target, expanding the end effector from an un-expanded state to an expanded state, and heating the end effector sufficiently so as to thermally treat tissue via conductive heating.

Certain embodiments of the present disclosure may include some, all, or none of the above aspects. Further, to the extent consistent, any of the aspects described herein may be used in conjunction with any or all of the other aspects described herein.

### BRIEF DESCRIPTION OF THE DRAWINGS

Various aspects and features of the present disclosure described herein with reference to the drawings wherein:

FIG. 1 is a schematic illustration of a gastrointestinal system of a patient, as seen in coronal view;

FIG. 2A is a perspective view of a surgical instrument provided in accordance with the present disclosure;

FIG. 2B is a schematic view of the surgical instrument of FIG. 1 deployed in a patient's colon;

FIG. 3 is a perspective view of the distal end of another surgical instrument provided in accordance with the present disclosure;

FIG. 4 is a perspective view of the distal end of yet another surgical instrument provided in accordance with the present disclosure;

FIG. 5 is a perspective view of the distal end of another surgical instrument provided in accordance with the present disclosure;

FIG. 6A is a perspective view of another surgical instrument provided in accordance with the present disclosure;

FIG. 6B is a schematic view of the surgical instrument of FIG. 6A deployed in a patient's colon;

FIG. 7 is a perspective view of the distal end of another surgical instrument provided in accordance with the present disclosure;

FIG. 8A is a perspective view of another surgical instrument provided in accordance with the present disclosure;

FIG. 8B is a schematic view of the surgical instrument of FIG. 8A deployed in a patient's colon.

### DETAILED DESCRIPTION

As IBD's such as ulcerative colitis may only affect portions of the colon, it is desirable to focus treatment towards such diseased areas while limiting damage to surrounding tissue and critical structures. Accordingly, the present disclosure provides various instruments and methods configured to facilitate the focused or controlled energy-based treatment of diseased portions of the colon while limiting damage to surrounding portions of the colon and other surrounding tissue and critical structures. The various embodiments of the present disclosure may be implemented using a variety of types of energy, e.g., RF, microwave, ultrasonic, thermal, etc. Further, although the various instruments and methods provided herein may be utilized to treat any suitable type of diseased tissue, particular reference will be made to the colon.

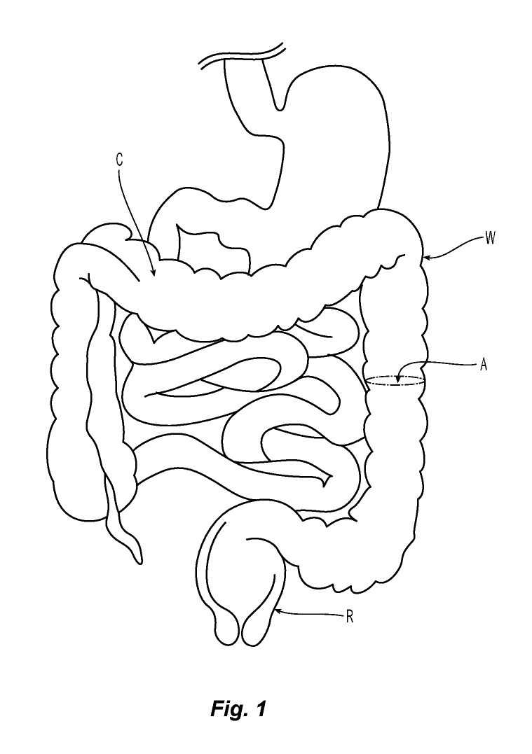

Referring to FIG. 1, a schematic view of a gastrointestinal system of a patient, generally, showing the stomach, small intestine, large intestine, colon “C”, and rectum “R.” The colon “C,” more specifically, has an inner wall “W” that defines an annular area “A,” terminating at the rectum “R.”

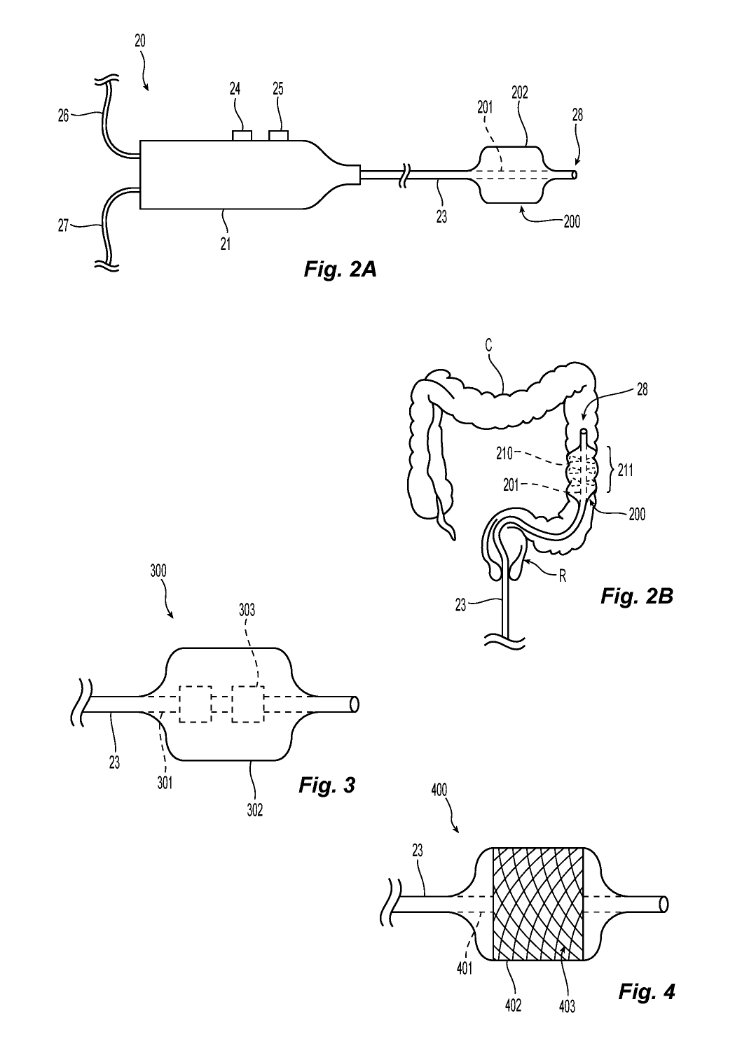

With reference to FIG. 2A, a surgical instrument provided in accordance with the present disclosure and configured to treat diseased bowel tissue in the colon “C” is shown generally identified as reference numeral 20. Surgical instrument 20, as described below, is configured for advancing the distal end through a bodily cavity, e.g. a human bowel, expanding from an un-expanded state to an expanded state to make contact with a treatment site, and providing heating or cooling at a treatment target to destroy mucosal tissue or other types of tissue, although use of surgical instrument 20 in various other surgical procedures is also contemplated and within the scope of the present disclosure. Surgical instrument 20 generally includes a housing 21, an end effector 200, a flexible tube 23, buttons 24, 25, a cable 26 adapted to connect to an energy source, e.g., a laser light source (not shown), and a supply line 27 adapted to connect to a fluid source (not shown).

Flexible tube 23 extends distally from housing 21 to end effector 200. End effector 200 includes probe 201 connected to flexible tube 23 and extending distally therefrom to distal end 28. End effector 200 further includes an inflatable balloon 202 composed of material that heats when laser light is applied, e.g. silicon, germanium, indium phosphate. Inflatable balloon 202 is disposed about probe 201 and configured to expand relative to probe 201 from the un-expanded state to the expanded state to conform to the interior anatomy of the colon “C.”

Button 25 controls the flow of fluid between supply line 27 and inflatable balloon 202. When button 25 is activated in a first position, fluid flows from the fluid source (not shown) through supply line 27 and flexible cable 23 into inflatable balloon 202 causing inflatable balloon 202 to inflate. When button 25 is activated in a second position, fluid flows from inflatable balloon 202 through flexible cable 23 and supply line 27 to the fluid source (not shown), causing inflatable balloon 202 to deflate.

Shaft 201 is configured to emit laser light 210 onto the interior surface of inflatable balloon 202, e.g., via apertures, slots, or other suitable openings formed in probe 201. The laser light 210 is configured to be absorbed by inflatable balloon 202 such that inflatable balloon 202 is heated. Button 24 controls the supply of laser light 210 to probe 201. When button 24 is activated, it allows laser light to travel from a laser light source (not shown) through cable 26 to probe 201. When button 24 is released or deactivated, it ceases to allow laser light 210 to be supplied to probe 201.

With reference to FIG. 2B, in use, distal end 28 of surgical instrument 20 is advanced into the rectum “R” and through the bowel “B” such that flexible tube 23 and end effector 200 are advanced through the colon “C” until end effector 200 reaches treatment target 211. Upon reaching treatment target 211, button 25 is activated to the first position to inflate balloon 202 to an expanded state in which inflatable balloon 202 conforms to the interior anatomy of treatment target 211, in circumferential contact therewith. Once inflatable balloon 202 has been inflated to contact treatment target 211, button 24 is activated to emit laser light 210 onto the interior surface of inflatable balloon 202 such that expandable balloon 202 absorbs the laser light 210 and is heated. Heating of inflatable balloon 202, in turn, conductively heats tissue in contact therewith, e.g., treatment target 211 on the colon wall “W.” Treatment target 211 is heated sufficiently, e.g., via controlling the application of laser light 210 to balloon 202, so as to treat the diseased tissue, e.g., via burning, charring, ablating, coagulating, and/or desiccating the diseased tissue. By thermally treating the tissue in this manner, inflammation and ulceration can be reduced or eliminated entirely, thereby reducing associated pain and discomfort. Further, during use, housing 21 may be manipulated to advance or retract end effector 200, while in an expanded state, through colon “C” to contact the entire length of treatment target 211 and treat the same with the heated balloon 202.

Referring to FIG. 3, another end effector 300 is provided in accordance with the present disclosure and configured for use with surgical instrument 20 (FIG. 2A). End effector 300 includes similar components and connections to surgical instrument 20 as end effector 200 (FIG. 2A), except as detailed below. End effector 300 includes a probe 301 that extends distally from flexible tube 23 of surgical instrument 20 (FIG. 2A). Probe 301 has an inflatable balloon 302 disposed thereabout that is formed from a material suitable for absorbing ultrasonic energy. One or more ultrasonic devices 303 (e.g., ultrasonic transducers) are coupled to probe 301 within inflatable balloon 302.

With additional reference to FIGS. 2A and 2B, end effector 300 may be utilized similarly as detailed above with respect to end effector 200. In use, distal end 28 of surgical instrument 20 is advanced into the rectum “R” and through the bowel “B” such that flexible tube 23 and end effector 300 are advanced through the colon “C” until end effector 300 reaches treatment target 211. Upon reaching treatment target 211, button 25 is activated in the first position to inflate inflatable balloon 302 to an expanded state in which inflatable balloon 302 conforms to the interior anatomy of treatment target 211, in circumferential contact therewith.

However, different from end effector 200, once balloon 302 is positioned as detailed above, button 24 may be activated to supply energy from a power source (not shown) connected to cable 26, through flexible tube 23, to ultrasonic devices 303. Ultrasonic devices 303 convert the energy into mechanical motion (e.g., ultrasonic vibrations) that are emitted from probe 301 within balloon 302 in the form of ultrasonic waves. These ultrasonic waves ultimately reach balloon 302 and are absorbed by balloon 302, causing balloon 302 to be heated. Similarly as above, heating of balloon 302 conductively heats treatment target 211 on the colon wall “W.” Treatment target 211 is heated sufficiently, e.g., via controlling the application of energy to ultrasonic devices 303, so as to treat the diseased tissue, e.g., via burning, charring, ablating, coagulating, and/or desiccating the diseased tissue. By thermally treating the tissue in this manner, inflammation and ulceration can be reduced or eliminated entirely, thereby reducing associated pain and discomfort. Further, during use, end effector 300 may be advanced or retracted through colon “C” to contact the entire length of treatment target 211 and treat the same with the heated balloon 302.

Referring to FIG. 4, another end effector 400 is provided in accordance with the present disclosure and configured for use with surgical instrument 20 (FIG. 2A). End effector 400 includes similar components and connections to surgical instrument 20 as end effector 200 (FIG. 2A), except as detailed below. End effector 400 includes a probe 401 that extends distally from flexible tube 23 and includes inflatable balloon 402 disposed thereabout. Inflatable balloon 402 includes a conductive coating 403, composed of, for example, Nichrome, Kanthal, PTC ceramics, an array of resistive heating elements, or other suitable materials and/or components capable of being resistively heated. Conductive coating 403 surrounds the outer periphery of inflatable balloon 402 and is connected through flexible tube 23 to cable 26 (FIG. 2A).

With additional reference to FIGS. 2A and 2B, end effector 400 may be utilized similarly as detailed above with respect to end effector 200. In use, distal end 28 of surgical instrument 20 is advanced into the rectum “R” and through the bowel “B” such that flexible tube 23 and end effector 400 are advanced through the colon “C” until end effector 400 reaches treatment target 211. Upon reaching treatment target 211, button 25 is activated to a first position to inflate inflatable balloon 402 to an expanded state in which inflatable balloon 402 conforms to the interior anatomy of treatment target 211, in circumferential contact therewith.

Once the above has been achieved, button 24 may be activated to supply energy from a power source (not shown) connected to cable 26, to conductive coating 403. Upon application of energy to conductive coating 403, conductive coating 403 is heated, thereby conductively heating tissue of the treatment target 211 in contact therewith. Treatment target 211 is heated sufficiently, e.g., via controlling the application of energy to conductive coating 403, so as to treat the diseased tissue, e.g., via burning, charring, ablating, coagulating, and/or desiccating the diseased tissue. By thermally treating the tissue in this manner, inflammation and ulceration can be reduced or eliminated entirely, thereby reducing associated pain and discomfort. Further, during use, end effector 400 may be advanced or retracted through colon “C” to contact the entire length of treatment target 211 and treat the same.

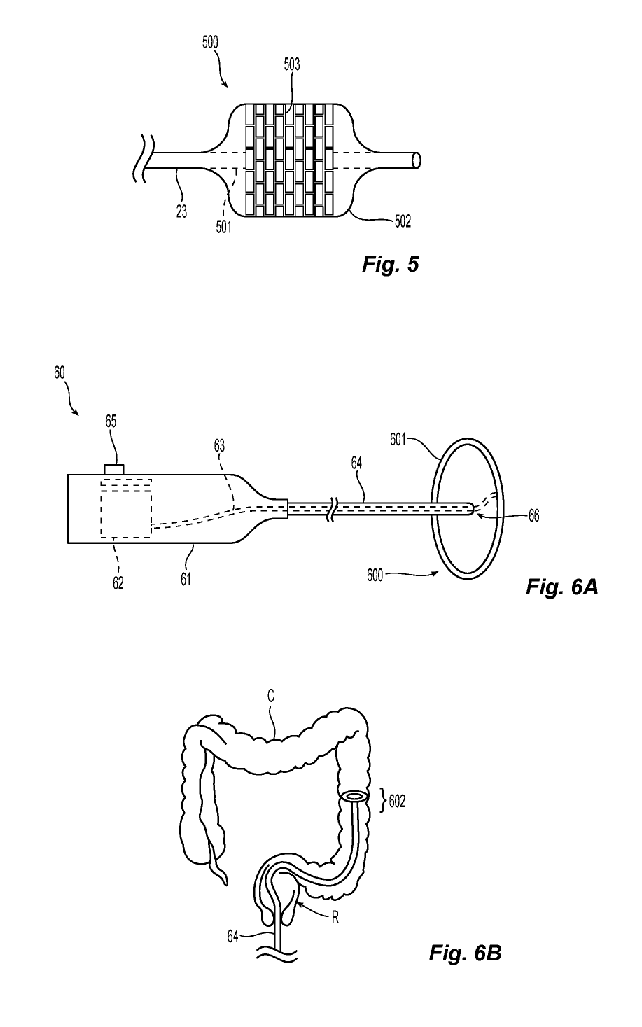

Referring to FIG. 5 another end effector 500 is provided in accordance with the present disclosure and configured for use with surgical instrument 20 (FIG. 2B). End effector 500 includes similar components and connections to surgical instrument 20 as end effector 200 (FIG. 2A), except as detailed below. End effector 500 includes a probe 501 that extends from flexible tube 23 and includes an inflatable balloon 502 disposed thereabout. End effector 500 additional includes a thermoelectric cooler 503 disposed about the exterior of inflatable balloon 502, and connected to cable 26 (FIG. 2B). Thermoelectric cooler 503 may be configured as a Peltier cooler formed from an array of parallel n-type and p-type semiconductor legs arranged such that the “hot side” of thermoelectric cooler 503 is oriented outwardly towards tissue and the “cold side” of the thermoelectric cooler 503 is oriented inwardly towards inflatable balloon 502. As such, upon supplying energy to thermoelectric cooler 503, heat is transferred outwardly across thermoelectric cooler 503, thereby cooling the “cold side” of thermoelectric cooler 503 and heating the “hot side” thereof.

With additional reference to FIGS. 2A and 2B, end effector 500 may be utilized similarly as detailed above with respect to end effector 200 (FIG. 2B). In use, distal end 28 of surgical instrument 20 is advanced into the rectum “R” and through the bowel “B” such that flexible tube 23 and end effector 500 are advanced through the colon “C” until end effector 500 reaches treatment target 211. Upon reaching treatment target 211, button 25 is activated to the first position thereof to inflate inflatable balloon 502 to an expanded state in which inflatable balloon 502 conforms to the interior anatomy of treatment target 211, in circumferential contact therewith.

Thereafter, button 24 is activated to supply energy from a power source (not shown), to thermoelectric cooler 503 via cable 26. Through the Peltier effect, as noted above, the outwardly-oriented “hot side” of thermoelectric cooler 503 is heated, thereby conductively heating the treatment target 211 in contact therewith. Treatment target 211 is heated sufficiently, e.g., via controlling the application of energy to thermoelectric cooler 503, so as to treat the diseased tissue, e.g., via burning, charring, ablating, coagulating, and/or desiccating the diseased tissue. By thermally treating the tissue in this manner, inflammation and ulceration can be reduced or eliminated entirely, thereby reducing associated pain and discomfort. Further, during use, end effector 500 may be advanced or retracted through colon “C” to contact the entire length of treatment target 211 and treat the same.

Referring to FIGS. 6A and 6B, another surgical instrument provided in accordance with the present disclosure and configured to treat diseased bowel tissue in the colon “C” is shown generally identified by reference numeral 60. Surgical instrument 60, as described below, is configured to be advanced into a bodily cavity, e.g. through the rectum “R” into the colon “C,” expanded from an un-expanded state to an expanded state to make contact with a treatment site, and provide heating at a treatment target to treat tissue, although use of surgical instrument 60 in various other surgical procedures is also contemplated and within the scope of the present disclosure.

Surgical instrument 60 generally includes a housing 61, an end effector 600, a flexible tube 64, a button 65, a fluid chamber 62, and a fluid line 63. Flexible tube 64 extends distally from housing 61 to end effector 600. End effector 600 includes an expandable ring 601 connected to fluid line 63 and radially disposed about the distal end of flexible tube 64. Expandable ring 601 is configured to expand from an un-expanded state to an expanded state to conform to the interior anatomy of the colon “C” in response to the delivery of fluid thereto. Fluid chamber 62 may define a closed-loop system, or may be coupled to an external fluid source (not shown) for the inflow and/or outflow of fluid to/from fluid chamber 62. Further, fluid chamber 62 may be configured to heat fluid disposed therein.

Button 65 controls the flow of fluid between fluid chamber 62 and expandable ring 601. When button 65 is activated, fluid is pumped from fluid chamber 62 through fluid line 63 into expandable ring 601 under sufficient pressure so as to cause expandable ring 601 to expand. Fluid may be continually circulated between fluid chamber 62 and expandable ring 601, with expandable ring 601 in the expanded state, to maintain the fluid at a desired temperature, e.g., via heating and re-circulating the fluid within fluid chamber 62. When button 65 is released or de-activated, fluid is no longer pumped into expandable ring 601 and, as such, expandable ring 601 is returned to the un-expanded state.

In use, distal end 66 of surgical instrument 60 is advanced into the rectum “R” and through the bowel “B” such that flexible tube 64 and end effector 600 are advanced through the colon “C” until end effector 600 reaches treatment target 602. Button 65 is activated such that the heated fluid within fluid chamber 62 is pumped through fluid line into expandable ring 601, causing expandable ring 601 to expand to the expanded state in which expandable ring 601 conforms to the interior anatomy of treatment target 602, in circumferential contact therewith. In addition to expanding expandable ring 601, the heated fluid also serves to heat expandable ring 601 which, in turn, heats the treatment target 602 on the colon wall “W” through conduction. Treatment target 602 is heated sufficiently, e.g., via controlling the heating of fluid within fluid chamber 62 and/or the circulation thereof through fluid line 63 and expandable ring 601, so as to treat the diseased tissue, e.g., via burning, charring, ablating, coagulating, and/or desiccating the diseased tissue. By thermally treating the tissue in this manner, inflammation and ulceration can be reduced or eliminated entirely, thereby reducing associated pain and discomfort. Further, during use, end effector 600 may be advanced or retracted through colon “C” to contact the entire length of treatment target 602 and treat the same.

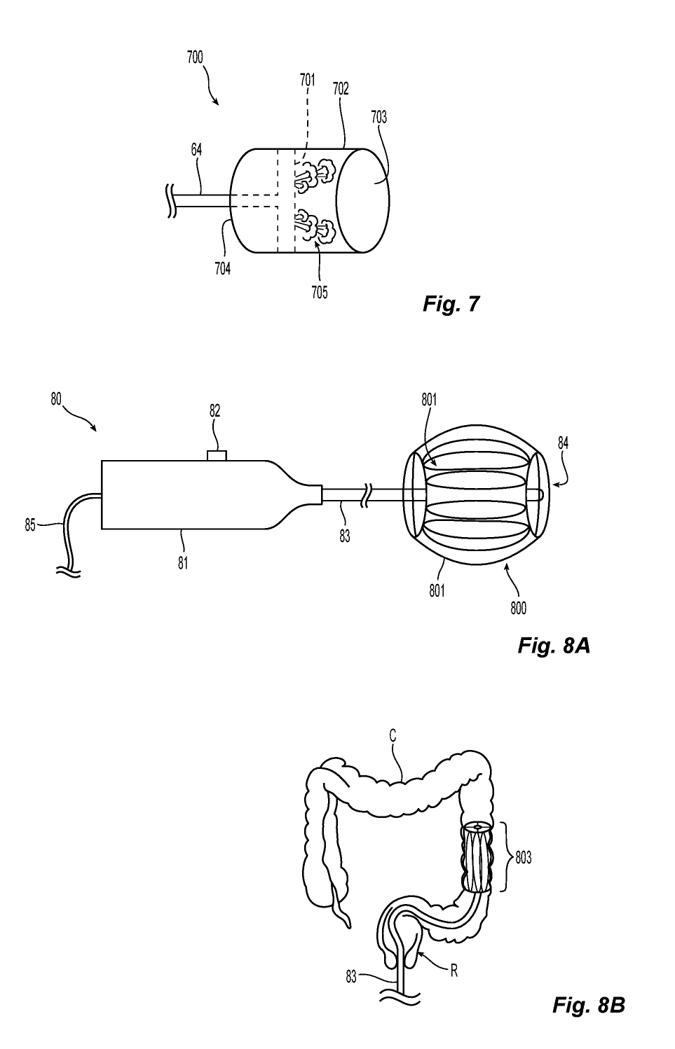

Referring to FIG. 7, another end effector 700 is provided in accordance with the present disclosure and configured for use with treatment device 60 (FIG. 6A). End effector 700 includes similar components and connections to surgical instrument 60 as end effector 600 (FIG. 6A), except as detailed below. End effector 700 includes a plunger 701 slidably disposed within flexible tube 64 and extending distally therefrom. End effector 700 further includes a cylinder 702 secured at the distal end of flexible tube 64 and defining a closed distal end 703 and a proximal end 704 that is closed about flexible tube 64. The head of plunger 701 extends into cylinder 702 and is slidably disposed within cylinder 702 in circumferential sealing engagement therewith. The head of plunger 701 is selectively translatable within cylinder 702 and configured to emit steam 705 into cylinder 702 on one side of the head of plunger 701, e.g., via slots, apertures, or other suitable openings facing distal end 703 of cylinder 702. As a result of this configuration, depending upon the positioning of plunger 701 within cylinder 702, the volume of cylinder 702 occupied by steam 705 may be varied. Plunger 701 may be coupled to a motor (not shown) disposed within housing 61 of surgical instrument 60 (FIG. 6A) or operably coupled thereto to enable sliding of the head of plunger 701 within cylinder 702, or may be manually movable therethrough.

With additional reference to FIGS. 6A and 6B, end effector 700 may be utilized similarly as detailed above with respect to end effector 600. Specifically, in use, distal end 66 of surgical instrument 60 is advanced into the rectum “R” and through the bowel “B” such that flexible tube 64 and end effector 700 are advanced through the colon “C” until end effector 700 reaches treatment target 602. More specifically, surgical instrument 60 is manipulated such that end effector 700 is positioned such that cylinder 702 is disposed adjacent the treatment target 602.

Once this position has been achieved, depending upon the size of the treatment target 602, the head of plunger 701 is advanced further into cylinder 702 towards distal end 703 thereof or retracted proximally within cylinder 702 towards proximal end 704 thereof. Thereafter, button 65 is activated to pump heated fluid, e.g., steam, from the fluid chamber 62 through plunger 701 and into the enclosed cylindrical volume between the head of plunger 701 and the closed distal end 703 of cylinder 702. The heated fluid, e.g., steam, within cylinder 702 conducts heat to cylinder 702 which, in turn, heat tissue in contact therewith and/or adjacent thereto to treat the diseased tissue, e.g., via burning, charring, ablating, coagulating, and/or desiccating the diseased tissue. By thermally treating the tissue in this manner, inflammation and ulceration can be reduced or eliminated entirely, thereby reducing associated pain and discomfort.

By changing the positioning of plunger 701 and, thus, the portion of cylinder 702 that is filled with the heated fluid, the treatment area can be broadened or narrowed. Further, during use, end effector 700 may be advanced or retracted through colon “C,” similarly as detailed above.

Referring to FIG. 8A, another surgical instrument provided in accordance with the present disclosure is shown generally identified by reference numeral 80. Surgical instrument 80, as described below, is configured for advancing the distal end through a bodily cavity, e.g., a human bowel, expanding from an un-expanded state to an expanded state to make contact with a treatment site, and heating a treatment target to treat tissue, although use of surgical instrument 80 in various other surgical procedures is also contemplated and within the scope of the present disclosure. Surgical instrument 80 generally includes a housing 81, an end effector 800, a flexible tube 83, a button 82, and a cable 85 connected to an energy source, e.g. an electrical outlet (not shown). Flexible tube 83 extends distally from housing 81 and supports end effector 800 at the free distal end thereof.

End effector 800 includes an expandable basket 801 formed from a plurality of wire components 802, at least some of which are coupled to one another via a hinge, e.g., a hinge joint or living hinge. Wire components 802 may be composed of, for example, Nichrome, Kanthal, or other suitable materials capable of conducting energy therethrough and, during such conduction of energy, being expanded and generating heat via resistive heating. As an alternative to energy-based expansion, expandable basket 801 may be formed from resilient wire components 802 and/or may include resilient hinges to bias expandable basket 801 towards the expanded position while also allowing collapse of expandable basket 801 for insertion, removal, and/or maneuvering expandable basket 801 into position.

Button 82 controls the flow of energy between a power source (not shown) connected to cable 85 and expandable basket 801. More specifically, when button 82 is activated, energy is supplied to expandable basket 801 and conducted through wire components 802 (including the hinges thereof) to expand expandable basket 801 to the expanded position. Thereafter or in connection therewith, energy being conducted through wire components 802 serves to resistively heat wire components 802. Upon release or deactivation of button 25, energy is no longer supplied to expandable basket 801 and, thus, expandable basket 801 is returned to the un-expanded state while wire components 802 are permitted to cool.

With additional reference to FIG. 8B, in use, distal end 84 of surgical instrument 80 is advanced into the rectum “R” and through bowel “B” such that flexible tube 83 and end effector 800 are advanced through the colon “C” until end effector 800 reaches treatment target 803. Upon reaching treatment target 803, button 82 is activated to supply energy to expandable basket 801, thereby expanding expandable basket 801, e.g., wherein wire components 802 bow outwardly to contact treatment target 803 about the full circumference thereof.

With wire components 802 in contact with treatment target 803, button 82 is maintained in the activated position such that the energy supplied to wire components 802 causes wire components 802 to be resistively heat and produce thermal energy that is conducted to treatment target 803 on the colon wall “W.” Treatment target 803 is heated sufficiently so as to treat the diseased tissue, e.g., via burning, charring, ablating, coagulating, and/or desiccating the diseased tissue. By thermally treating the tissue in this manner, inflammation and ulceration can be reduced or eliminated entirely, thereby reducing associated pain and discomfort. Housing 81 may be manipulated to advance or retract end effector 800, while in the expanded state, through colon “C” to contact the entire length of treatment target 803.

From the foregoing and with reference to the various figure drawings, those skilled in the art will appreciate that certain modifications may also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical instrument, comprising:
a flexible tube defining a proximal end and a distal end; and
an end effector disposed at the distal end of the flexible tube, the end effector configured for insertion into and advancement through a body cavity to a treatment target, the end effector adapted to connect to a source of energy and including:
an expandable member expandable from an un-expanded state to an expanded state for circumferentially contacting a treatment target; and
at least one heating member configured to heat the expandable member such that the expandable member is capable of thermally treating tissue in contact therewith.

2. The surgical instrument according to claim 1, wherein the expandable member is an inflatable balloon.

3. The surgical instrument according to claim 2, wherein the at least one heating member includes a laser source disposed within the inflatable balloon and configured to emit laser light onto the inflatable balloon, the inflatable balloon configured to absorb the emitted laser light, thereby heating the inflatable balloon.

4. The surgical instrument according to claim 2, wherein the at least one heating member includes at least one ultrasonic device disposed within the inflatable balloon, the at least one ultrasonic device configured to emit ultrasonic waves onto the inflatable balloon, the inflatable balloon configured to absorb the ultrasonic waves, thereby heating the inflatable balloon.

5. The surgical instrument according to claim 2, wherein the at least one heating member includes a thermoelectric cooler disposed about the inflatable balloon.

6. The surgical instrument according to claim 2, wherein the at least one heating member includes a conductive coating disposed about the inflatable balloon, the conductive coating configured for resistive heating in response to application of energy thereto.

7. The surgical instrument according to claim 1, wherein the expandable member includes an expandable ring.

8. The surgical instrument according to claim 7, wherein the at least one heating member includes a fluid chamber configured to heat fluid therein and pump the heated fluid into the expandable ring to both expand the expandable ring and heat the expandable ring.

9. The surgical instrument according to claim 1, wherein the expandable member includes an expandable basket and wherein the at least one heating member includes a plurality of wire components forming at least a portion of the expandable basket, the plurality of wire components configured for resistive heating in response to conduction of energy therethrough.

10. The surgical instrument according to claim 9, wherein the expandable basket further includes at least one joint interconnecting at least two of the plurality of wire components, and wherein the at least one joint is configured to flex, thereby expanding the expandable basket, in response to conduction of energy therethrough.

11. A method of treating tissue at a treatment target, comprising:
inserting a flexible tube having an end effector disposed at a distal end thereof into a body cavity;
positioning the end effector adjacent a treatment target;
expanding the end effector from an un-expanded state to an expanded state; and
heating the end effector sufficiently so as to thermally treat tissue in contact therewith via conductive heating.

12. The method according to claim 11, wherein the end effector includes an inflatable balloon, and wherein expanding the end effector from the un-expanded state to the expanded state includes inflating the inflatable balloon.

13. The method according to claim 12, wherein heating the end effector includes emitting laser light onto an interior of the inflatable balloon such that the inflatable balloon absorbs the emitted laser light, thereby heating the inflatable balloon.

14. The method according to claim 12, wherein heating the end effector includes emitting ultrasonic waves onto an interior of the inflatable balloon, such that the inflatable balloon absorbs the emitted ultrasonic waves, thereby heating the inflatable balloon.

15. The method according to claim 12, wherein the end effector includes a thermoelectric cooler disposed about the inflatable balloon, and wherein heating the end effector includes supplying energy to the thermoelectric cooler.

16. The method according to claim 12, wherein the end effector includes a conductive coating disposed about the inflatable balloon, and wherein heating the end effector includes supplying energy to the conductive coating to resistively heat the conductive coating.

17. The method according to claim 11, wherein the end effector includes an expandable ring and wherein heated fluid is pumped into the expandable ring to both expand the expandable ring and heat the expandable ring.

18. The method according to claim 11, wherein the end effector includes an expandable basket having a plurality of wire components and wherein the end effector is both expanded and heating via conducting energy through the plurality of wire components.
